(19) United States
(12) Patent Application Publication
(10) Pub. No.: US 2024/0225467 A1
(43) Pub. Date: Jul. 11, 2024

(54) NON-CONTACT BLOOD FLOW OBSERVATION APPARATUS

(71) Applicant: LEIMAC LTD., Moriyama-shi, Shiga (JP)

(72) Inventors: Takunori SHIMAZAKI, Kawanishi (JP); Yoshifumi KAWAKUBO, Awa (JP); Yuhei HAYASHI, Moriyama (JP); Jun MITSUDO, Moriyama (JP); Jumpei OHUCHI, Moriyama (JP)

(21) Appl. No.: 18/559,792

(22) PCT Filed: Feb. 15, 2022
(86) PCT No.: PCT/JP2022/005788

§ 371 Date: Nov. 8, 2023

(30) Foreign Application Priority Data
Jan. 21, 2022 (JP) 2022-008228

(51) Int. Cl. A61B 5/026; A61B 5/00; G06T 7/00

(52) U.S. Cl. CPC A61B 5/0261; A61B 5/004; A61B 5/7425; G06T 7/0012; G06T 2207/10004; G06T 2207/10016; G06T 2207/10024; G06T 2207/30104

(57) ABSTRACT

Provided is a non-contact blood flow observation apparatus that enables speedy and clear observation of states of blood flow in a contactless manner. This non-contact blood flow observation apparatus 1 includes an image acquirer 2 that acquires a first image 2im that is a moving image or a series of still images of a region including a blood vessel, and an image processor 3 that correlates a different color with each gradation of brightness in advance, creates a second image 3im by assigning the color corresponding to the brightness for each singular or plurality of pixels in the first image 2im, and displays the second image 3im that is a moving image or a series of still images on a display device 5.

### TECHNICAL FIELD

The present invention relates to a non-contact blood flow observation apparatus that enables states of blood flow to be observed contactlessly.

### BACKGROUND ART

It is not unusual for states of blood vessels to be observed using various types of observing apparatuses, besides general visual examination, palpation, and so forth. Among such observing apparatuses, there are known observing apparatuses in which light is cast on a region including blood vessels that are objects, and images are observed, as disclosed in Japanese Patent Application Laid-open No. 2012-152459, Japanese Patent Application Laid-open No. 2013-150712, and WO 2017/051455. Such apparatuses perform observation without touching the human body (i.e., contactlessly), and accordingly enable infection risk to be suppressed and also speedy observation to be performed.

### BRIEF SUMMARY

However, the observation apparatuses disclosed in the patent documents cited above go no further than to impart contrast to an image of blood vessels with different depths from the skin, and further technological development as an observation apparatus is possible.

The present invention has been made in light of the foregoing situation, and accordingly it is an object thereof to provide a non-contact blood flow observation apparatus that enables speedy and clear observation of states of blood flow in a contactless manner.

In order to achieve the above object, a non-contact blood flow observation apparatus according to an embodiment of the present invention includes an image acquirer that acquires a first image that is a moving image or a series of still images of a region including a blood vessel, and an image processor that correlates a different color with each gradation of brightness in advance, creates a second image by assigning the color corresponding to the brightness for each singular or plurality of pixels in the first image, and displays the second image that is a moving image or a series of still images on a display device.

Preferably, the color corresponding to the gradation of the brightness is blue, green, red, and a mixed color thereof. Preferably, further, the color when the brightness is highest is one of blue and red, and the color when the brightness is lowest is the other of blue and red.

Preferably, the image processor displays the second image on the display device at the same time as the first image, with a screen split.

According to the non-contact blood flow observation apparatus of the present invention, speedy and clear observation of states of blood flow in a contactless manner is enabled.

### BRIEF DESCRIPTION OF DRAWINGS

FIG. 1 is a schematic diagram illustrating a usage example of a non-contact blood flow observation apparatus according to an embodiment of the present invention.

FIG. 2 is a photograph showing an example of a first image acquired by an image acquirer of the above non-contact blood flow observation apparatus.

FIG. 3 is a schematic diagram illustrating an image processor realized by a computer system in the above non-contact blood flow observation apparatus.

FIGS. 4A-4D are diagrams showing an example of colors corresponding to brightness in the image processor in the above non-contact blood flow observation apparatus.

FIGS. 5A-5D are photographic examples of a second image created by the image processor of the above non-contact blood flow observation apparatus.

FIGS. 6A-6D are photographic examples in which arrows indicating a position of a sample pixel in FIGS. 5A-5D are added.

FIG. 7 is a graph showing temporal change in brightness at the sample pixel in FIG. 6.

FIG. 8 is a diagram showing an example of the image processor of the above non-contact blood flow observation apparatus displaying, on a display device, the second image at the same time as the first image, with a screen split.

### DETAILED DESCRIPTION

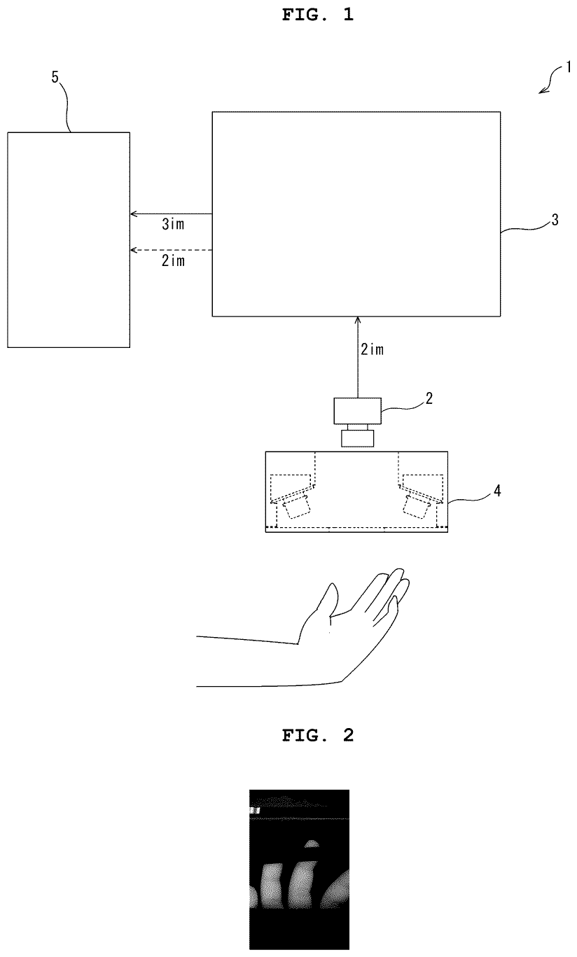

An embodiment for carrying out the present invention will be described below. A non-contact blood flow observation apparatus 1 according to the embodiment of the present invention includes an image acquirer 2 and an image processor 3, as illustrated in FIG. 1.

The image acquirer 2 is for performing image-capturing of a region including a blood vessel, thereby acquiring a first image 2im that is a moving image or a series of still images. The region including the blood vessel can be a vicinity of a site that will receive or has received medical treatment, a site that will receive or has received treatment of a blood vessel for the purpose of medical treatment (and the vicinity thereof), or the like.

The region including the blood vessel is not limited in particular, and can be the entire body. FIG. 2 shows an example of the first image 2im when the region including the blood vessel is fingers of a hand.

In order to acquire the first image 2im that is clear using the image acquirer 2, light can be cast by a light irradiator 4 (a ring illuminator in FIG. 1) on the region including the blood vessel. The light can be selected as appropriate such that the first image 2im that is clear can be acquired, examples thereof being white, green, blue, or the like. A commonplace illuminator, such as a shadowless lamp or the like, may be used for the light irradiator 4.

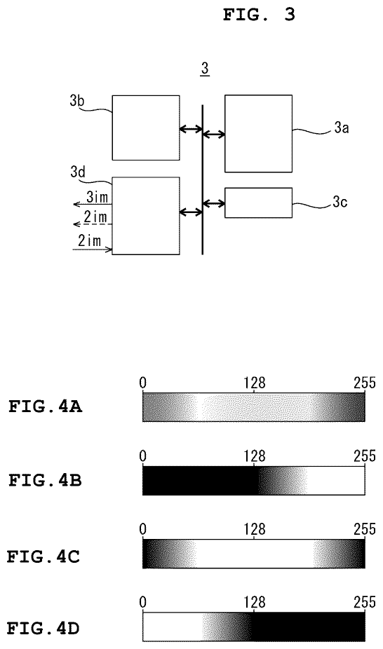

The image processor 3 processes the first image 2im sent from the image acquirer 2. The image processor 3 normally is realized by a computer system as illustrated in FIG. 3, and has a program for performing image processing in program memory 3a. In FIG. 3, sign 3b denotes a CPU, sign 3c denotes work memory, and sign 3d denotes other portions including an input/output unit.

The image processor 3 correlates, in advance, a different color for each gradation of brightness of the first image 2im. Gradations are, for example, 256 gradations or the like. Examples of colors corresponding to the gradations of brightness can include blue, green, red, and mixed colors thereof. The color can be set to be blue when the brightness is the highest, and can be set to be red when the brightness is the lowest. Conversely, the color can be set to be red when the brightness is the highest, and can be set to be blue when the brightness is the lowest. In these cases, the color in the middle of brightness is green. Between blue and green are mixed colors thereof, and between green and red are mixed colors thereof. This is because with such an arrangement, the order of blue, green, and red is in the order of wavelength of light, and is generally readily understood.

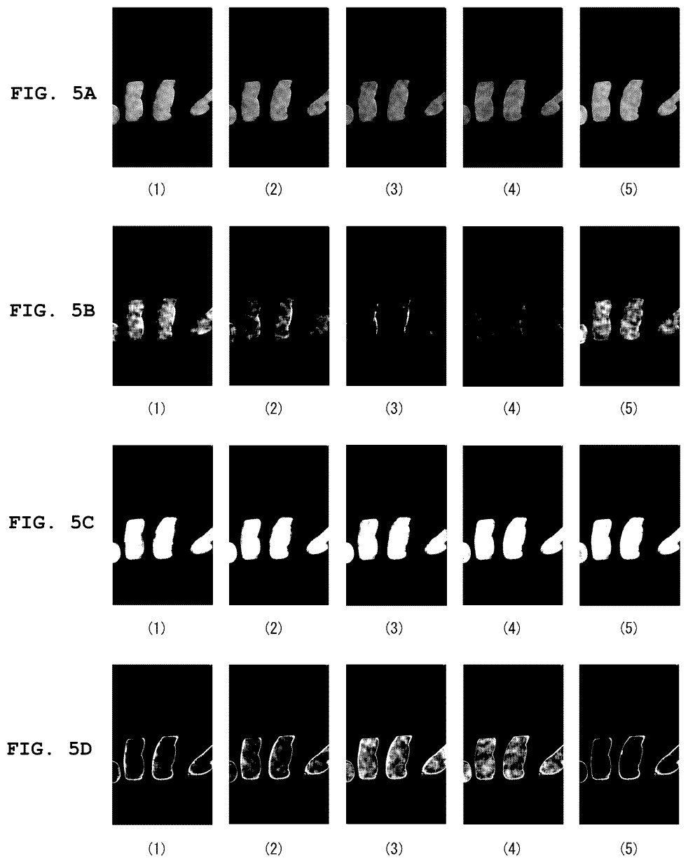

FIG. 4A shows a conversion into grayscale, in an arrangement in which the colors corresponding to the gradations of brightness are blue, green, red, and mixed colors thereof, the color when the brightness is the highest (indicated by numeral 255 in the Figure) is blue, the color when the brightness is the lowest (indicated by numeral 0 in the Figure) is red, and the color when the brightness is the middle (indicated by numeral 128 in the Figure) is green. FIGS. 4B, 4C, and 4D show the blue component, the green component, and the red component in FIG. 4A. FIGS. 4B, 4C, and 4D are to facilitate the original color, since FIG. 4A is shown in grayscale due to circumstances regarding illustration as a Figure.

The image processor 3 assigns each pixel in the first image 2im a color correlated in advance with the brightness, thereby creating a second image 3im. Alternatively, in some cases, the image processor 3 can assign each of a plurality of pixels in the first image 2im a color correlated in advance with the brightness (an averaged brightness, a median value brightness, or the like), thereby creating the second image 3im. The image processor 3 then displays the second image 3im that is a moving image or a series of still images on a display device 5.

In the second image 3im that is a moving image or a series of still images, the color changes in the portions of blood vessels in accordance with the state of the blood flow (more specifically, in accordance with change in the amount of hemoglobin, change in the shape of the blood vessels, and so forth). There is hardly any change in color in portions where no blood vessels are present. Accordingly, the non-contact blood flow observation apparatus 1 enables contactless observation of the state of blood flow, speedily and clearly. Also, the non-contact blood flow observation apparatus 1 can perform contactless observation of the state of blood flow, thereby suppressing infection risk, and also there is no need for work that affects the human body, such as injecting contrast dye or the like.

FIG. 5A is an example of second images 3im when the region including the blood vessel is fingers of a hand, and shows a conversion into grayscale at five separate times (indicated by (1), (2), (3), (4), and (5) in the Figure) in a moving image or a series of still images. FIGS. 5B, 5C, and 5D show the blue component, the green component, and the red component in FIG. 5A. FIGS. 5B, 5C, and 5D are to facilitate the original color, since FIG. 5A is shown in grayscale due to circumstances regarding illustration as a Figure.

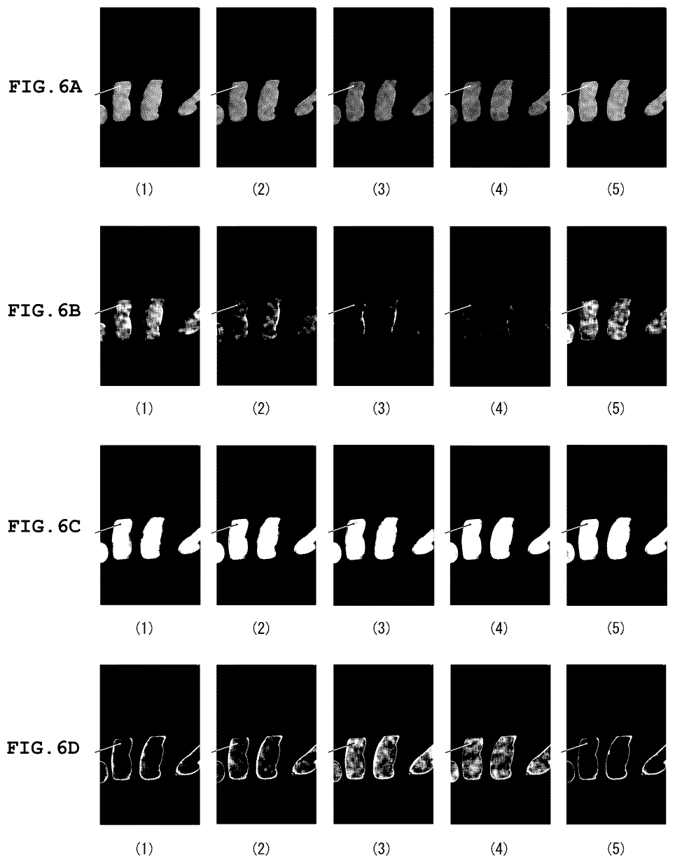

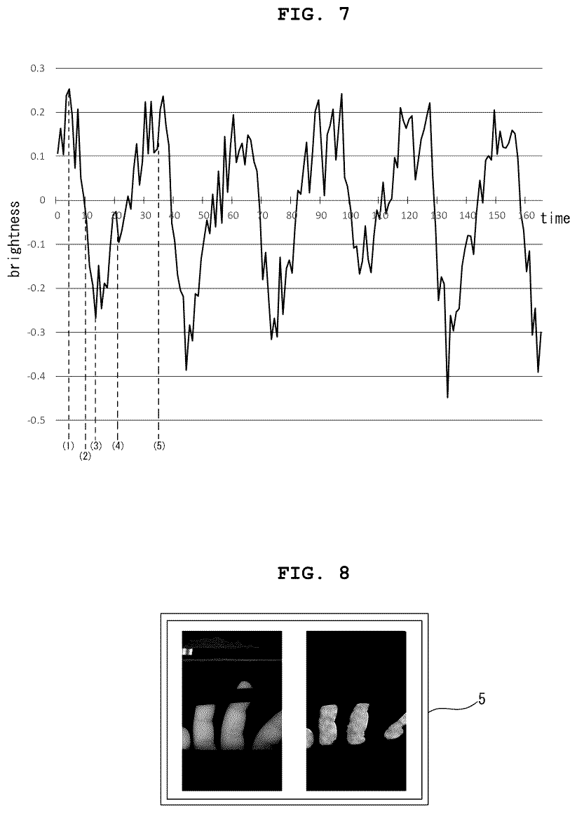

FIGS. 6A to 6D are each the same Figures as FIGS. 5A to 5D, in which arrows indicate a sample pixel. FIG. 7 is a graph showing temporal change in brightness at the sample pixel. The (1), (2), (3), (4), and (5) in FIG. 7 indicate the times (1), (2), (3), (4), and (5) in FIGS. 5A to 5D (and in FIGS. 6A to 6D). Note that the vertical axis and the horizontal axis in FIG. 7 are those prior to conversion into actual brightness values and time, and accordingly no units have been listed.

It can be understood from FIG. 7 that the brightness changes in accordance with pulsation. This change in brightness in FIG. 7 corresponds to the change in color of the sample pixel shown in FIGS. 6A to 6D. Specifically, according to FIGS. 6B to 6D, the green component is greatest at any of times (1) to (5), but some blue component is present at time (1), the blue component is no longer present at time (2), no blue component is present and some red component is present at time (3), there is less red component at time (4), and no red component is present and some blue component is present at time (5).

Also, the pixels making up the second image 3im are displayed at positions corresponding to the pixels making up the first image 2im, and accordingly, the image processor 3 displaying the second image 3im on the display device 5 at the same time as the first image 2im with a screen split facilitates understanding of which portion of the first image 2im has changed, as shown in FIG. 8.

The entire non-contact blood flow observation apparatus 1 can also be integrated, and a terminal equipped with a camera, such as a smartphone, a laptop computer, or the like, for example, can be used.

Although the non-contact blood flow observation apparatus according to an embodiment of the present invention has been described above, the present invention is not limited to the description in the embodiment described above, and various design modifications can be made within the scope of matters set forth in the Claims.

### REFERENCE SIGNS LIST

1 Non-contact blood flow observation apparatus
2 Image acquirer
2im First image
3 Image processor
3a Program memory
3b CPU
3c Work memory
3d Other portions of computer system
3im Second image
4 Light irradiator
5 Display device

1. A non-contact blood flow observation apparatus, comprising:
an image acquirer that acquires a first image that is a moving image or a series of still images of a region including a blood vessel in a vicinity of a site that will receive or has received medical treatment, or in a vicinity of a site that will receive or has received treatment of a blood vessel for medical treatment; and
an image processor that correlates a different color with each gradation of brightness in advance, creates a second image by assigning the color corresponding to the brightness for each singular pixel in the first image, and displays change in the color, corresponding to change in the brightness in accordance with pulsation, in the second image, the second image being a moving image or a series of still images on a display device.

2. The non-contact blood flow observation apparatus according to claim 1, wherein
the color corresponding to the gradation of the brightness is blue, green, red, and a mixed color thereof.

3. The non-contact blood flow observation apparatus according to claim 2, wherein
the color when the brightness is highest is one of blue and red, and the color when the brightness is lowest is the other of blue and red.

4. The non-contact blood flow observation apparatus according to claim 1, wherein
the image processor displays the second image on the display device at the same time as the first image, with a screen split.
